(19) United States
(12) Patent Application Publication
(10) Pub. No.: US 2012/0214198 A1
(43) Pub. Date: Aug. 23, 2012

(54) ALGACULTURE METHOD

(75) Inventor: Walter Trösch, Stuggart (DE)

(73) Assignee: FRAUNHOFER-GESELLSCHAFT ZUR FORDERUNG DER ANGEWANDTEN FORSCHUNG E.V., Munich (DE)

(21) Appl. No.: 13/503,348

(22) PCT Filed: Oct. 15, 2010
(86) PCT No.: PCT/EP2010/006314

§ 371 Date: Apr. 20, 2012

(30) Foreign Application Priority Data
Oct. 20, 2009 (DE) 10 2009 051 588.7

(51) Int. Cl. C12N 1/12; C12P 1/00; C12P 13/04; C12M 1/12; C12P 33/00; C12P 21/00; C12P 19/04; C12M 1/00; C02F 3/32; C12P 7/64

(52) U.S. Cl. 435/52; 210/602; 435/257.1; 435/257.3; 435/41; 435/106; 435/134; 435/71.1; 435/101; 435/292.1

(57) ABSTRACT

The present invention relates to bioprocess engineering methods in which aqueous phases of anaerobic biologically treated organic suspensions are supplied to algal cultures as media components. The present invention also relates to the use of aqueous phases of anaerobic biologically treated organic suspensions as media components of algal cultures. Additionally, the present invention relates to the use of aqueous phases of anaerobic biologically treated organic suspensions to improve the growth conditions of algae in photobioreactors. Furthermore, the present invention relates to the use of algae for the treatment of aqueous phases of anaerobic biologically treated organic suspensions, in particular of anaerobic biologically treated sewage filtrate. The present invention also relates to bioprocess engineering devices (100) comprising a bioreactor (1), in particular a fermentation tower and a photobioreactor (3).

The present invention relates to the further purification of aqueous phases of anaerobic biologically treated organic suspensions. Moreover, the present invention relates to the cultivation of algae, in particular microalgae.

The present invention relates to bioprocess engineering methods in which aqueous phases of anaerobic biologically treated organic suspensions are supplied to algal cultures as media components. Moreover, the present invention relates to methods according to the invention designed as manufacturing methods. The present invention also relates to the use of aqueous phases of anaerobic biologically treated organic suspensions as media components of algal cultures. Additionally, the present invention relates to the use of aqueous phases of anaerobic biologically treated organic suspensions to improve the growth conditions of algae in photobioreactors. Furthermore, the present invention relates to the use of algae for the treatment of aqueous phases of anaerobic biologically treated organic suspensions, in particular of anaerobic biologically treated sewage filtrate. The present invention also relates to bioprocess engineering devices comprising a bioreactor, in particular a fermentation tower, and a photobioreactor as well as the use of said devices for the methods according to the invention.

Devices and methods for the treatment of sewage, especially of municipal wastewater are known. Examples include so-called sewage treatment plants or wastewater treatment plants. They are primarily used for the treatment of municipal wastewater which is collected in the public sewerage system and transported to the sewage treatment plant. The purpose of wastewater treatment is the removal of unwanted components from the wastewater in order to obtain treated wastewater which can for example be released into streams and bodies of water (so-called receiving bodies of water) without posing a risk to the environment and the population caused by chemical or microbiological exposure.

Mechanical (physical), biological and chemical methods, usually in combination, are used to remove the unwanted components. Accordingly, modern sewage treatment plants comprise at least three stages, wherein at least one physical treatment stage, at least one biological treatment stage and at least one chemical treatment stage are connected in series.

Biological methods are mainly used to breakdown organic compounds in wastewaters with high organic exposure. This is commonly achieved by means of aerobic breakdown of the organic compounds into inorganic end products such as carbon dioxide, nitrate, sulfate, phosphate, etc. For this purpose, biomass is used in the form of activated sludge in so-called stirred tanks or as bacterial lawn in the form of so-called bacteria beds. Not only are the organic compounds to be removed utilized energetically and “respirated,” i.e. broken down through conversion with atmospheric oxygen by way of catabolic processes, but they also promote the growth of the biomass in anabolic processes. The accumulating biomass regularly needs to be removed from the biological treatment stage and subject to after-treatment such as fermentation in a fermentation tower, wherein biogas is released while considerable amounts of sludge waste still remain. This sludge waste can partly be utilized as fertilizer. However, for the most part the accumulated sludge is disposed of in incinerators in a costly process.

Anaerobic biological wastewater treatment is another known procedure. It also serves the removal of harmful or interfering organic carbon compounds in the wastewater by means of microbiological breakdown processes which however take place without oxygen. In the process, the anaerobic microorganisms obtain the energy required for their metabolism from the conversion of the organic carbon compounds and convert them into organic acids, other hydrocarbons and into carbon dioxide and methane. The wastewater treatment takes place in hermetically sealed reactors.

Anaerobic methods are generally divided into flow systems without immobilization of the biomass, wherein the biomass is essentially provided in suspension, and into flow systems with immobilization, wherein the biomass is essentially provided immobilized on inner structures of the reactor with the wastewater to be treated flowing around it.

One known example is the so-called “Emscher fountain,” a two-story digestion tower patented by Immhoff in 1906, which allows anaerobic fermentation in the lower fermentation compartment.

Aside from fermentation towers, fluidized reactors, anaerobic stirred tanks, UASB reactors or fixed-bed reactors are also used for the known anaerobic wastewater treatment.

However, anaerobic treatment is not only used for municipal wastewater and sewage sludge, but also for other wastewater, for example wastewater from industry, in particular the food industry, or wastewater from the fermentation of renewable raw materials. For example, the processing of sugar beets in sugar refineries produces highly contaminated wastewater which is treated by means of known anaerobic sewage treatment plants. In the process, the wastewater is broken down in two stages: in a first stage, the so-called acidification, all the sugars of high molecular weight and other organic compounds present in the wastewater are broken down into organic acids by microorganisms. These acids are the degradable substrate for bacteria in the second stage, the so-called methanogenic phase. The methanogenic phase is the microbial phase in which the wastewater is treated by converting the COD (dissolved oxidizable organic substance) into the gaseous products CH₄ and CO₂, which leave the water as such, at no cost and voluntarily.

The residual concentrations of organic contaminations achieved with known anaerobic treatment methods do not allow the direct discharge into the receiving body of water. Therefore, further aerobic biological, physical and/or chemical treatment stages are normally required.

Methods and devices used for the anaerobic biological treatment of wastewater, wherein biogas can simultaneously be harvested are disclosed for example in DE 10 2005 063 228 A1.

The anaerobically treated wastewater also contains biomass, in particular bacteria, at a concentration which does not allow the direct discharge into the receiving body of water. Therefore, said biomass is often removed from the wastewater by means of physical methods, in particular by means of filtration. Separation processes in particular by means of membranes are used for this filtration. Separation stages of the membrane separation technique can be classified by separation limits into micro-, ultra- and nanofiltration as well as reverse osmosis. During the separation process, the biomass accumulates as solid matter at least directly on the membrane, while the filtered fluid passes through the membrane.

Different filter systems for the after-treatment of anaerobically treated wastewater are known. DE 100 04 096 A1 and DE 10154549 A1 for example describe filter systems which are suitable for microfiltration or ultrafiltration of anaerobically treated wastewater. Commonly, dynamic filtration is used in which a concentrate (retentate) and a filtrate (permeate) are obtained during a thickening process. The concentrate contains the suspended, dispersed or emulgated particles as well as the dissolved substances corresponding to the separation capability of the selected filter system, for example the selected membrane. The concentrate is commonly returned to the anaerobic treatment stage. The filtrate is the treated fluid, free of particles which are larger than the separation limit of the filter system.

Although the treatment efficacy in connection with the breakdown of organic contaminations is high with these kinds of anaerobic biological treatments of wastewater, in particular in combination with a subsequent filtration, the wastewater treated in this fashion still contains high amounts of phosphate and nitrogen. Therefore, an environmentally friendly disposal of these wastewaters is necessary in order to prevent the receiving body of water from being exposed to large amounts of phosphate and nitrogen.

The anaerobic treatment of wastewater results in the production of biogas. The anaerobic treatment of wastewater generates large amounts of CO₂. Large amounts of methane are also obtained which can be burned to produce energy and generates more CO₂. In order to reduce or even prevent the release of CO₂ in amounts that are harmful to the environment, it is consequently necessary to either reduce the production of CO₂ or to retain or convert the generated CO₂ in an environmentally friendly fashion. Photosynthesis is one of the few natural processes in which CO₂ is consumed. Derived from CO₂, water and light as energy source, CO₂ is bonded in plants and algae, thus producing water and oxygen. Close to 360 Gt of CO₂ are bonded globally every year by way of photosynthesis. In the oceans, microalgae bond nearly 175 Gt of CO₂. These microalgae in the oceans are the basis of the food chain.

The photosynthetic biomass production can significantly contribute to the reduction of the atmospheric CO₂ content, if it is used both as carbon storage and as substitute for fossil fuels or fossil raw materials. Because during the energetic utilization of photosynthetically produced biomass only the same amount of CO₂ is released as was photosynthetically bonded during the growth phase, CO₂ is saved, provided that the process is sustainable, if a product manufactured with fossil raw materials is replaced.

Because of their small size and fine distribution, algae, in particular microalgae, are characterized by greater photosynthetic efficiency compared to higher plants. Said better utilization of available sunlight results in higher biomass yields compared to land plants. The biomass productivity of microalgae in photobioreactors, which is associated with a higher CO₂ consumption, is up to ten times higher than in land plants. Moreover, microalgae are capable of producing a multitude of high-quality lipophilic or hydrophilic substances, including vitamins, color pigments, fatty acids, amino acids and pharmaceuticals, for instance antibiotics. Important substance classes include for example essential fatty acids, lipids, sterols and carotenoids, polysaccharides, proteins or amino acids and phycobiliproteins (pigments) as well as the total biomass as protein-rich, low-nucleic acid value raw material. These products can be used as high-quality foods, food additives, animal food, pharmaceuticals or substitutes for synthetic substances in the cosmetic and chemical industry.

Microalgae include the prokaryotic cyanobacteria on the one hand as well as eukaryotic microscopic classes of algae.

Compared to higher land plants, the microalgae biomass is characterized by the absence of lignin, a low cellulose and nucleic acid content and a high carbohydrate and protein ratio of up to 60% of the dry substance.

The utilization of CO₂ as waste product is a basic condition for the production of microalgae with sunlight under net energy generation conditions, so that a material-related energetic or even a purely energetic utilization of algal biomass becomes sustainable. This helps contribute to the reduction of atmospheric CO₂ generated by the burning of fossil energy carriers. Corresponding attempts are currently under way in some pilot plants in connection with the burning of different fossil energy carriers.

Like any other plants, microalgae also require valuable minerals for their growth in addition to solar energy and CO₂, such as nitrogen and phosphorus as well as other elements, for example trace elements. They are normally provided in the form of synthetic culture media. In some cases, liquid fertilizer for agricultural products is used for this purpose, which is tailored to the needs of flowers and cultivated plants. The mineral components represent a significant factor of the production-related operating costs for the cultivation of microalgae, because sunlight and CO₂ are available at no cost.

The profitability of biomass produced by microalgae in general and the specific substances in particular is initially defined by the productivity of the selected species of algae. However, this is only true if a high conversion rate of solar irradiation energy into the desired form of biomass is simultaneously achieved and the energetic expenses and costs for the manufacture, installation and operation of the system are kept extremely low. High biomass productivity is a question of optimal light distribution per volume. The absorption of light by algae results in a strong decrease in light as the layer thickness increases, while mutual self-shading takes place at the same time. Photobioreactors are capable of achieving adequate conversion efficiency rates of the solar irradiation energy. Photobioreactors are fermenters used to cultivate phototrophic microorganisms, in particular algae, cyanobacteria, and purple bacteria, i.e. which either enable the growth and propagation of these cells or promote the production of different substances by means of phototrophic cells. A photobioreactor which is particularly suitable for the cultivation of microalgae is disclosed in DE 199 16 597 A1.

One of the technical problems of the present invention consists in the provision of methods, in particular economical methods, for the removal of large amounts of nitrogen and phosphorus from anaerobic biologically treated wastewater.

The invention was also based on the technical problem of providing economical and simple culture media for algae and microalgae, in particular for the cultivation in photobioreactors.

The invention was also based on the technical problem aimed at providing methods and devices which make it possible to utilize, in particular utilize in an energetically favorable manner, the breakdown products obtained by way of anaerobic treatment of wastewater and organic waste.

The present invention was also based on the technical problem aimed at providing methods and devices which allow an energetically favorable cycle of anaerobic breakdown of organic compounds and photosynthetic build-up of organic compounds.

The present invention was also based on the problem aimed at utilizing breakdown products generated by the treatment of wastewater, in particular the aqueous phases of biologically treated organic suspensions in an ecologically and economically meaningful manner.

The present invention solves the technical problem it is based upon in particular also by providing a method, in particular a bioprocess engineering method, comprising the step: supply of at least one aqueous phase of at least one anaerobic biologically treated organic suspension as media component to an algal culture.

In other words, the present invention in particular also solves the technical problem it is based upon by providing a bioprocess engineering method in which at least one aqueous phase of at least one anaerobic biologically treated suspension is supplied as media component to an algal culture, in particular microalgal culture, in a main process step.

The term “main process step” refers to the process step of supplying the at least one aqueous phase of at least one anaerobic biologically treated organic suspension as media component to an algal culture which is crucial for the present invention.

In addition to this main process step, other process steps can also be provided in the method according to the invention. These additional process steps can take place before or after the main process step, i.e. they can be provided as process steps upstream or downstream of the main process step.

According to the invention, an aqueous phase of an anaerobic biologically treated organic suspension is preferably supplied as media component to an algal culture.

In the context of the present invention, the term bioprocess engineering method refers to any method in which biological and/or biochemical process steps are used for the conversion and/or production of substances.

In the context of the present invention, an anaerobic biologically treated organic suspension is an anaerobic biologically treated suspension which contains organic components.

In the context of the present invention, an anaerobic biologically treated organic suspension is a heterogeneous mixture of substances consisting of a fluid, for example water, containing finely distributed, in particular organic solid matter, wherein the suspension or at least the solid, in particular organic components, of the suspension were subject to anaerobic biological treatment. A fluid, in particular water, can be added before, during or after said treatment in order to obtain a suspension.

In the context of the present invention, an aqueous phase of the anaerobic biologically treated organic suspensions is a liquid part of said suspensions which can mainly contain water and substances dissolved therein, but also other fluids. The aqueous phase can be obtained by way of separation from the solid phase or from a mixture of fluid and solid matter (concentrate), for example by means of filtration. Consequently, the aqueous phase can contain part of the fluid or the entire fluid of the anaerobic biologically treated organic suspensions.

In a preferred embodiment, the concentrate extracted from the aqueous phase is not used as media component of the algal culture. This means that the concentrates are preferably not supplied to the algal culture. In a preferred embodiment, the aqueous phase is not further treated by means of an aerobic trickling filter.

A preferred embodiment of the aqueous phase is a filtrate. In a preferred embodiment, the at least one filtrate is supplied as media component to the algal culture without an additional interim step, in particular without an additional treatment step.

In a preferred embodiment, the filtrate contains less than 1% by weight of solid matter. In a preferred embodiment, the filtrate only contains solid matter in dissolved format. In a preferred embodiment, the filtrate contains less than 1% by weight of solid matter, wherein the solid matter is exclusively present in the filtrate in dissolved format.

In a preferred embodiment, the aqueous phase is extracted from the anaerobic biologically treated organic suspension in an additional step before being supplied to the algal culture. This means that an extraction step precedes the main step in a preferred embodiment, in which the aqueous phase is extracted from the other components of the anaerobic biologically treated organic suspension. A physical extraction is preferred according to the invention.

A preferred embodiment of the aqueous phase is a filtrate. However, alternative process steps for the extraction of an aqueous phase from an anaerobic biologically treated organic suspension are also possible, in particular physical methods, such as for example the decantation of the aqueous phase.

According to the invention, the anaerobic biological treatment is preferably an anaerobic fermentation.

In a preferred embodiment, the aqueous phase is derived from a methane fermentation process.

In an embodiment, the anaerobic biological treatment has already been performed before the method according to the invention is carried out. In this case, the anaerobic biological treatment is not a process step of the method according to the invention. In an embodiment, the anaerobic biological treatment has already been performed before the method according to the invention is carried out. In this case, the anaerobic biological treatment is not a process step of the method according to the invention. [sic] In an alternative embodiment, the anaerobic biological treatment is a process step of the method according to the invention which precedes the main process step.

In a preferred embodiment, the anaerobic biological treatment is performed in an additional step as single-stage or two-stage methanization before the aqueous phase is supplied to the algal culture. In an embodiment according to the invention, the anaerobic biological treatment is performed in an additional step as single-stage methanization before the aqueous phase is supplied to the algal culture. In an embodiment according to the invention, the anaerobic biological treatment is performed in an additional step as two-stage methanization before the aqueous phase is supplied to the algal culture. In an embodiment according to the invention, the anaerobic biological treatment is performed in an additional step as multi-stage methanization before the aqueous phase is supplied to the algal culture.

In an embodiment according to the invention, the anaerobic biological treatment is performed in an additional step as methanization cascade before the aqueous phase is supplied to the algal culture. Hence, the methanization is preferably conducted in bioreactors connected in series, for example in fermentation towers.

This means that in an embodiment according to the invention, in a step a) the organic suspension is anaerobic biologically treated and in a step b) the aqueous phase of the anaerobic biologically treated organic suspension is supplied as media component to an algal culture.

According to the invention, the original, in particular untreated, organic suspensions are preferably sewage sludge, organic waste, wastewater from the food industry, effluent from municipal wastewater and/or wastewater from the fermentation of renewable raw materials. According to the invention, the original, in particular untreated, organic suspensions are preferably sewage sludge, organic waste and/or wastewater. According to the invention, the original, in particular untreated, suspensions are preferably sewage sludge and/or wastewater.

According to the invention, the aqueous phase is preferably a filtrate, i.e. a filtered, clear fluid. According to the invention, the aqueous phase is preferably a filtrate of the anaerobic biologically treated organic suspension. According to the invention, the aqueous phase is preferably a sterile filtrate.

According to the invention, the anaerobic biologically treated organic suspension and/or the aqueous phase is preferably filtered at least once by means of a filter in an additional step before being supplied to the algal culture. In an alternative embodiment according to the invention, the anaerobic biologically treated organic suspension is filtered at least once by means of a filter in an additional step before being supplied to the algal culture. In an alternative embodiment according to the invention, the aqueous phase is filtered at least once by means of a filter in an additional step before being supplied to the algal culture, for example if the aqueous phase was obtained by means of decanting.

This means that in an embodiment according to the invention, in a step a) the organic suspension is anaerobic biologically treated, in a step b) the anaerobic biologically treated organic suspension is filtered and in a step c) the aqueous phase of the anaerobic biologically treated suspension obtained by means of filtration is supplied as media component to an algal culture.

In an embodiment according to the invention, the anaerobic biologically treated organic suspension and/or the aqueous phase are filtered at least once by means of at least one filtration device, in particular microfiltration device, selected from a rotating disk filter, a polymer filter and a ceramic depth filter in an additional step before being supplied to the algal culture.

In an embodiment according to the invention, the anaerobic biologically treated organic suspension and/or the aqueous phase are dynamically filtered at least once by means of at least one filtration device, in particular microfiltration device, selected from a rotating disk filter, a polymer filter and a ceramic depth filter in an additional step before being supplied to the algal culture.

In an embodiment according to the invention, the anaerobic biologically treated organic suspension and/or the aqueous phase are filtered at least once by means of a rotating disk filter, a polymer filter or a ceramic depth filter in an additional step before being supplied to the algal culture. In an embodiment according to the invention, the anaerobic biologically treated organic suspension and/or the aqueous phase is filtered at least once by means of a rotating disk filter in an additional step before being supplied to the algal culture. According to the invention, a rotating disk filter, a polymer filter or a ceramic depth filter is preferably used for the filtration. In an embodiment according to the invention, a rotating disk filter is used for the filtration.

In an embodiment according to the invention, a polymer filter is used for the filtration. In an embodiment according to the invention, a ceramic depth filter is used for the filtration. In an embodiment according to the invention, a dynamic rotating disk filter is used for the filtration. In an embodiment according to the invention, a dynamic polymer filter is used for the filtration. In an embodiment according to the invention, a dynamic ceramic depth filter is used for the filtration.

In an embodiment according to the invention, a dynamic filtration method is used.

In an embodiment according to the invention, the filter is a ceramic filter or a polymer membrane. According to the invention, the filter is preferably a ceramic filter.

In an embodiment according to the invention, the filter is a device described in DE 101 54 549 A1. The content disclosed in DE 101 54 549 A1 is fully integrated into the present description of the invention. In an embodiment according to the invention, the filter is a device for separating substances, in particular solid matter, liquid phases of different density and/or gases, from a fluid by way of rotation with a plurality of filter elements which allow the filtered fluid to pass and which are retained in a case pivotable around a rotational axis, said case comprising an inlet opening for the fluid, at least one outlet opening for the separated heavy substances and at least one outlet opening for the filtered fluid, wherein a passage for substances with a lower density than the fluid is provided in the area or close to the rotational axis of the filter elements.

In an embodiment according to the invention, the anaerobic biologically treated organic suspension and/or the at least one aqueous phase is filtered in an additional step before being supplied to the algal culture. In an embodiment according to the invention, the anaerobic biologically treated organic suspension and/or the at least one aqueous phase is microfiltered or ultrafiltered in an additional step before being supplied to the algal culture. In an embodiment according to the invention, the anaerobic biologically treated organic suspension and/or the at least one aqueous phase is microfiltered in an additional step before being supplied to the algal culture. In an embodiment according to the invention, the anaerobic biologically treated organic suspension and/or the at least one aqueous phase is ultrafiltered in an additional step before being supplied to the algal culture.

The filtration is used in particular for the extraction of possible organic solid matter, in particular for the extraction of microorganisms. This prevents the contamination of the algal culture with other microorganisms. Such contamination could impair the algal growth on the one hand and require a treatment of the algal culture on the other hand, in order to be able to dump the wastewater of the algal culture into the ocean. Furthermore, the treatment of substances derived from algae might become more difficult.

In an embodiment according to the invention, it can be provided that the concentrates extracted from the filtrate during the filtration, also known as fermentation residues, are resupplied to an anaerobic biological treatment step.

In an embodiment according to the invention, the concentrates are viscous.

According to the invention, the aqueous phase is preferably free of fecal bacteria. In an embodiment of the invention, the aqueous phase is completely or almost free of bacteria. According to the invention, the aqueous phase is therefore preferably a filtrate free of fecal bacteria, in particular a sterile filtrate. According to the invention, the aqueous phase is preferably free or almost free of fungi. According to the invention, the aqueous phase is preferably free or almost free of viruses. This helps ensure a low-germ microalgae biomass production through which the microalgae biomass can be supplied to high-quality recycling.

It has surprisingly been shown that the aqueous phases can be treated smoothly in such a way that they do not contain any bacteria or at least only contain bacteria in concentrations so low that the bacteria neither impair the algal growth nor are harmful to the health. In particular, it was surprisingly demonstrated that filtrates of anaerobic biologically treated organic suspensions are virtually or even completely free of bacteria.

Moreover, it has been shown that the aqueous phases, in particular filtrates, contain toxic substances from the fermentation process in concentrations so low that they are not impairing the algal growth.

It has surprisingly been shown that aqueous phases derived from anaerobic biologically treated organic suspensions can be used as substitute and partial substitute of media, for example mineral salt media for the cultivation of algae, in particular microalgae. The use of aqueous phases derived from anaerobic biologically treated organic suspensions as media components surprisingly results in higher accrual rates of the algae biomass than the use of traditional synthetic media which feature an excellent provision of nitrogen.

Consequently, the addition of nitrogen to algal cultures which was achieved with the time-consuming, costly and energy-consuming Haber-Bosch process can be reduced with the method according to the invention or it may even be possible that none of said nitrogen is required for the cultivation of algae.

It was surprisingly demonstrated that aqueous phases derived from anaerobic biologically treated organic suspensions supply adequate if not ideal amounts of valuable minerals, in particular nitrogen and phosphorus, to the algae thanks to their composition. Without being bound to the theory, it is assumed that not only the ammonium levels present in the aqueous phases, but also the available carbonate quantities help stimulate the algal growth.

According to the invention, the aqueous phase of the anaerobic biologically treated organic suspension preferably contains 0.5 to 5 g/L of ammonium ions, in particular 1 to 3 g/L of ammonium ions.

According to the invention, the aqueous phase of the anaerobic biologically treated organic suspension preferably contains between 1 and 500 mg/L of phosphate, in particular between 10 and 200 mg/L of phosphate. In an embodiment according to the invention, the aqueous phase of the anaerobic biologically treated organic suspension contains at least 1 mg/L of phosphate. In an embodiment according to the invention, the aqueous phase of the anaerobic biologically treated organic suspension contains at least 10 mg/L of phosphate. In an embodiment according to the invention, the aqueous phase of the anaerobic biologically treated organic suspension contains at most 500 mg/L of phosphate. In an embodiment according to the invention, the aqueous phase of the anaerobic biologically treated organic suspension contains at most 200 mg/L of phosphate.

Furthermore, it has been shown that the COD (chemical oxygen demand) in the aqueous phases can be reduced further as a result of the facultative heterotrophic metabolism of algae.

Hence, the bioprocess engineering method according to the invention can for example in particular comprise the following steps:
a) anaerobic biological treatment of an organic suspension,
b) filtration of the anaerobic biologically treated organic suspension, and
c) supply of the filtrate derived from the anaerobic biologically treated organic suspension as media component to an algal culture.

In the context of the present invention, a media component of an algal culture refers to a certain ratio of a fluid composition, which is suitable for the cultivation of algae, in particular microalgae. The ratio can range from 1% to 100% of the total algal culture medium. The person skilled in the art is familiar with different algal culture media from the prior art.

According to the invention, the total medium used for the algal culture preferably contains at least 50% by weight of the aqueous phase as media component.

According to the invention, the total medium used for the algal culture preferably contains at least 50% by weight of the aqueous phases. According to the invention, the total medium used for the algal culture preferably contains at least 60% by weight of the aqueous phases. In an embodiment according to the invention, the total medium used for the algal culture contains at least 75% by weight of the aqueous phases. In an embodiment according to the invention, the total medium used for the algal culture contains at least 80% by weight of the aqueous phases. In an embodiment according to the invention, the total medium used for the algal culture contains at least 90% by weight of the aqueous phases. The total medium supplied to the algal culture can consist up to 99.9% of the aqueous phase. Moreover, the total medium used for the algal culture can consist of the at least one aqueous phase. It is also possible that the total medium used for the algal culture consists exclusively of the aqueous phases.

According to the invention, it can therefore be provided that traditional algal culture medium is completely or partly replaced with the aqueous phase. According to the invention, it can therefore be provided that traditional algal culture medium is completely replaced with aqueous phase. According to the invention, it can therefore be provided that the traditional algal culture medium is replaced partly with the aqueous phase, for example at a ratio of 50%, of 75% or of 90%.

Since the composition of an aqueous phase derived from an anaerobic biologically treated organic suspension depends on the composition of the starting suspension, for example on the wastewater composition, the growth stimulation can be further improved as needed by adding trace elements.

According to the invention, it can also be provided that trace elements are added to the at least one aqueous phase. According to the invention, it can also be provided that phosphorus, for example in the form of phosphate, is added to the at least one aqueous phase.

Analogous to the composition of the mineral salt medium which guarantees maximum product growth rates for a specific alga species, the difference of ingredients, in particular phosphorus, can be balanced out between the aqueous phases used as medium according to the invention and optimal growth media in an embodiment according to the invention.

Moreover, it can be provided according to the invention that the ratio between phosphorus and nitrogen is adjusted to about 3 to 1, for example ranging between 2 to 1 and 4 to 1, in particular 3 to 1, in the at least one aqueous phase. The adjustment can be achieved by adding phosphorus or nitrogen.

According to the invention, the algal culture is preferably a microalgal culture.

According to the invention, the algal culture is preferably a Phaeodactylum tricornutum culture, or a Haematococcus pluvialis culture, or a Chlorella sorokiniana culture, or a Chlorella vulgaris culture, or a Platymonas subcordiformis culture, or a Tetraselmis suecica culture, or a Nannochloropsis oculata culture, or an lsochrysis sp. culture, or a Nannochloropsis limnetica culture, or a Phormidium sp. culture, or a Pseudoanabaena sp. culture, or a Dunaliella sp. culture, or a Monodus subterraneus culture.

In an embodiment according to the invention, the algal culture is a Phaeodactylum tricornutum culture. In an embodiment according to the invention, the algal culture is a Haematococcus pluvialis culture. In an embodiment according to the invention, the algal culture is a Chlorella sorokiniana culture. In an embodiment according to the invention, the algal culture is a Chlorella vulgaris culture. In an embodiment according to the invention, the algal culture is a Platymonas subcordiformis culture. In an embodiment according to the invention, the algal culture is a Tetraselmis suecica culture. In an embodiment according to the invention, the algal culture is a Nannochloropsis oculata culture. In an embodiment according to the invention, the algal culture is an lsochrysis sp. culture. In an embodiment according to the invention, the algal culture is a Nannochloropsis limnetica culture. In an embodiment according to the invention, the algal culture is a Phormidium sp. culture. In an embodiment according to the invention, the algal culture is a Pseudoanabaena sp. culture. In an embodiment according to the invention, the algal culture is a Dunaliella sp. culture. In an embodiment according to the invention, the algal culture is a Monodus subterraneus culture.

In an embodiment according to the invention, the algal culture is used to produce valuable substances, in particular essential fatty acids, lipids, sterols and carotenoids, polysaccharides, proteins or amino acids and phycobiliproteins.

Furthermore, the algal culture can be used to obtain, i.e. produce biomass as raw material. In the process, the biomass is created by the growing mass of algae. Moreover, the algal culture can be used to obtain protein-rich, low nucleic acid value biomass as raw material. The algal culture can also be used to obtain carbohydrate-rich, low nucleic acid value biomass as raw material.

In an embodiment according to the invention, the algal culture, in particular the algal culture in a photobioreactor, can be used as raw material for a biomass refinery. In an embodiment according to the invention, it can be provided that the algal culture in a photobioreactor is used as raw material for a biomass refinery, in which at least one valuable substance, in particular at least one valuable substance selected from the group comprising essential fatty acids, lipids, sterols, carotenoids, polysaccharides, proteins, amino acids, phycobiliproteins or mixtures thereof, is produced as a first product and biomass is produced as a second product.

According to the invention, 1 to 10% of valuable substance and 90 to 99% of biomass are preferably produced in the process, in particular 4 to 6% of valuable substance and 94 to 96% of biomass.

As a result, the algal cultures cultivated according to the invention can achieve a surprisingly good ratio of valuable substance to biomass.

In a preferable embodiment according to the invention, the valuable substance is at least one omega-3 fatty acid, in particular one omega-3 fatty acid. The valuable substance can in particular be eicosapentaenoic acid (EPA). However, the valuable substance can for example also be docosahexaenoic acid (DHA). In an alternative embodiment, the valuable substance can also be a carotenoid, in particular lutein.

Accordingly, in an embodiment according to the invention, it can be provided that the aqueous phase consisting of at least one anaerobic biologically treated organic suspension, for example wastewater or sewage sludge, is used as starting material for the method according to the invention, and a valuable substance is obtained as product, for example EPA or lutein.

Hence, the invention also relates to a method according to the invention, in particular a bioprocess engineering method, in which at least one aqueous phase consisting of at least one anaerobic biologically treated organic suspension is supplied as media component to an algal culture in a main process step, for the manufacture of biomass and/or at least one valuable substance.

Therefore, the invention also relates to a method according to the invention designed as a manufacturing method.

The invention also relates to a method according to the invention for the manufacture of biomass from an algal culture and/or the manufacture of valuable substances. In other words, the invention also relates to a method according to the invention for the manufacture of biomass from an algal culture. The invention thus also relates to a method according to the invention for the manufacture of valuable substances.

The invention also relates to biomasses and/or valuable substances manufactured by means of the method according to the invention.

The algal culture can also be used for the further purification of aqueous phases of anaerobic biologically treated organic suspensions. The algal culture can be used in particular for the reduction of nitrogen, phosphorus and/or the chemical oxygen demand (COD) in aqueous phases of anaerobic biologically treated organic suspensions.

The invention consequently also relates to a method according to the invention, in particular a bioprocess engineering method, in which at least one aqueous phase consisting of at least one anaerobic biologically treated organic suspension is supplied as media component to an algal culture in a main process step, for the manufacture of treated organic suspensions with a reduced nitrogen, phosphorus and/or COD content.

Moreover, the invention relates to a method according to the invention for the treatment of aqueous phases of anaerobic biologically treated organic suspension.

The aqueous phases of anaerobic biologically treated organic suspension can be used to improve the growth conditions of algae, in particular of microalgae in photobioreactors. The aqueous phases of anaerobic biologically treated organic suspension can also be used to reduce substrate-related operating costs associated with the cultivation of algae, in particular microalgae in photobioreactors.

The algal culture can also be used to bind free CO₂.

In an embodiment of the invention, it can be provided that not only the aqueous phase consisting of at least one anaerobic biologically treated organic suspension is supplied as media component to the algal culture, but that additionally also the CO₂ generated in connection with the anaerobic biological treatment of the organic suspension is supplied to the algal culture. The bioprocess engineering method according to the invention can therefore for example in particular comprise the following steps:
a) anaerobic biological treatment of an organic suspension,
b) filtration of the anaerobic biologically treated organic suspension,
c) supply of the filtrate derived from the anaerobic biologically treated organic suspension as media component to an algal culture,
d) supply of the CO₂ generated in connection with the anaerobic biological treatment to the algal culture.

Optionally, the methane generated in connection with the anaerobic biological treatment can be burned to generate energy and the CO₂ generated in connection with the incineration can also be supplied to the algal culture.

Therefore, the bioprocess engineering method according to the invention can for example in particular comprise the following steps:
a) anaerobic biological treatment of an organic suspension,
b) filtration of the anaerobic biologically treated organic suspension,
c) supply of the filtrate derived from the anaerobic biologically treated organic suspension as media component to an algal culture,
d) supply of the CO₂ generated in connection with the anaerobic biological treatment to an algal culture,
e) burning of the methane generated in connection with the anaerobic biological treatment to generate energy,
f) supply of the CO₂ generated in connection with the methane incineration to the algal culture.

Consequently, the bioprocess engineering method according to the invention can for example in particular comprise the following steps:
a) anaerobic biological treatment of an organic suspension,
b) filtration of the anaerobic biologically treated organic suspension,
c) supply of the filtrate derived from the anaerobic biologically treated organic suspension as media component to an algal culture,
d) burning of the biogas generated in connection with the anaerobic biological treatment to generate energy,
e) supply of the CO₂ generated in connection with the methane incineration to the algal culture.

The present invention therefore provides a method which allows the recirculation of a CO₂-absorbing process. The end products produced in the preceding process step of the inorganic biological treatment include an aqueous phase and CO₂ associated with the fermentation and incineration of the generated methane and the generated biomass. In the subsequent process step, namely the cultivation of algae, these products, i.e. the aqueous phase and CO₂ can be used as starting materials, in particular also used completely. By cultivating the algae, these starting materials optionally produce a valuable substance along in particular with biomass. Said biomass in turn can be used as starting material, wherein an aqueous phase and CO₂ are produced after the energetic exploitation and subsequent treatment. Consequently, the method according to the invention can be used in a cycle in which generated energy and valuable substances can be diverted.

Furthermore, the method according to the invention helps save operating costs for the cultivation of algae, in particular in photobioreactors, because the starting materials CO₂ and nutrients in the form of the aqueous phase result from the anaerobic biological treatment of the organic suspension. This makes it possible for example to use wastewater generated at a factory in purified form as culture media for algal cultures, while the CO₂ also generated at the factory as a result of the incineration process can again be supplied to said algal cultures.

This allows an economical and at the same time environmentally friendly use of the aqueous phases of anaerobic biologically treated organic suspensions, in particular of wastewater or sewage sludge.

According to the invention, the algal culture is preferably cultivated in a photobioreactor.

The person skilled in the art is familiar with photobioreactors suitable for the cultivation of algae, in particular microalgae.

In an embodiment according to the invention, the photobioreactor is a photobioreactor described in DE 199 16 597 A1. The content disclosed in DE 199 16 597 A1 is fully integrated into the present description of the invention.

According to the invention, a photobioreactor is preferable which has a reactor chamber made of translucent material. According to the invention, a photobioreactor is preferable whose reactor chamber has a surface enlargement that is greater than the straight-surfaced enveloping area of a volume. According to the invention, a photobioreactor is preferable which has appliances for a turbulent flow control. According to the invention, a photobioreactor is preferable which has elements used to direct light from the outside into the reactor chamber.

The present invention also relates to the use of aqueous phases of anaerobic biologically treated organic suspensions as media component of an algal culture, in particular a microalgal culture.

The present invention also relates to the use of aqueous phases of anaerobic biologically treated organic suspensions to improve the growth conditions of algae, in particular microalgae, in photobioreactors.

Moreover, the present invention relates to the use of algae, in particular of microalgae, for the treatment of aqueous phases of anaerobic biologically treated organic suspensions, in particular of anaerobic biologically treated wastewater filtrate.

The present invention also relates to the products obtained with the cultivation of algae according to the invention.

Additionally, the present invention relates to a bioprocess engineering device, comprising a bioreactor, for example a fermentation tower, and a photobioreactor.

The bioreactor is in particular a reactor suitable for the anaerobic biological treatment of suspensions, i.e. an anaerobic reactor.

According to the invention, the device preferably comprises a filtration device.

According to the invention, the filtration device is a microfiltration device.

According to the invention, the filtration device or the microfiltration device preferably comprises means to remove solid matter.

According to the invention, the bioreactor preferably comprises a supply line to the filtration device and the filtration device comprises a supply line to the photobioreactor.

According to the invention, the bioreactor preferably comprises a fluid supply line to the filtration device. According to the invention, the filtration device preferably comprises a fluid supply line to the photobioreactor. According to the invention, the filtration device preferably comprises a supply line to the bioreactor to transfer concentrates, also known as fermentation residues, extracted from the filtrate in the filtration device. According to the invention, it is preferable that the filtration device alternatively or in particular additionally comprises a discharge line for the concentrates, i.e. fermentation residues.

A preferred embodiment is a bioprocess engineering device, comprising a bioreactor, a filtration device and a photobioreactor, wherein the bioreactor comprises a fluid supply line to the filtration device and the filtration device comprises a fluid supply line to the photobioreactor.

A preferred embodiment is a bioprocess engineering device, comprising a bioreactor, a filtration device and a photobioreactor, wherein the bioreactor comprises a fluid supply line to the filtration device and the filtration device comprises a fluid line to the photobioreactor and wherein the filtration device comprises means to remove solid matter and/or wherein the filtration device comprises a solid matter supply line to the bioreactor to transfer the concentrates.

According to the invention, the bioreactor preferably comprises a gas discharge line to discharge gases generated in connection with the fermentation, for example biogas, methane and/or CO₂.

According to the invention, the photobioreactor preferably comprises a gas supply line to supply CO₂.

In an embodiment according to the invention, the device comprises a combustion chamber for burning the biogas or methane accumulated in the bioreactor. According to the invention, the combustion chamber is preferably connected with the bioreactor by way of the gas discharge line and with the photobioreactor by way of the gas supply line. According to the invention, the burning of gases in the combustion chamber is preferably used to generate energy.

According to the invention, the bioreactor preferably comprises devices for mixing the reactor content.

The present invention also relates to a special design of the bioprocess engineering device according to the invention used to conduct the method according to the invention.

Furthermore, the invention relates to the use of a bioprocess engineering device according to the invention used to conduct the method according to the invention.

Present descriptions of the alternative and/or preferred embodiments according to the invention of the method according to the invention relate to the embodiments of the uses according to the invention, products according to the invention and devices according to the invention.

Present descriptions concerning alternative and/or preferred embodiments according to the invention of the methods according to the invention also relate to embodiments of methods according to the invention, products according to the invention and devices according to the invention.

Present descriptions concerning alternative and/or preferred embodiments according to the invention of the devices according to the invention also relate to embodiments of the uses according to the invention, products according to the invention and methods according to the invention.

Further embodiments of the present invention can be found in the subclaims.

The invention is explained in more detail by means of the following example and figures:

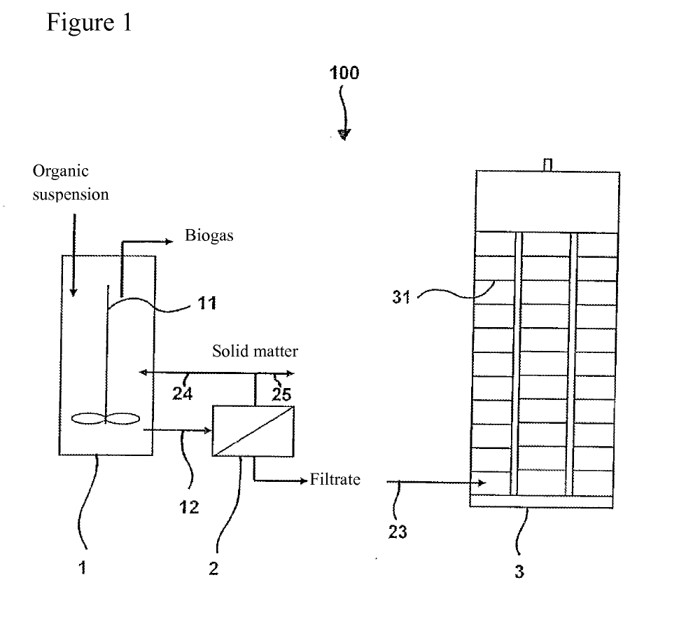

FIG. 1 illustrates an embodiment of a device according to the invention, comprising a bioreactor, a filtration device and a photobioreactor.

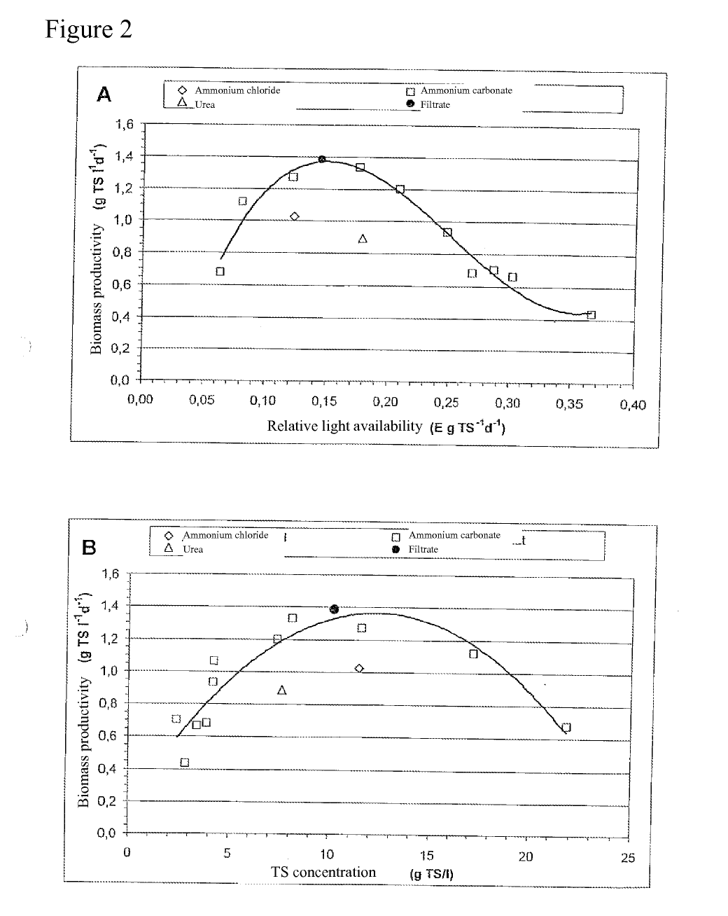

FIG. 2 illustrates comparisons of the biomass productivity of Phaeodactylum tricornutum with different N-sources depending on the relative light availability (FIG. 2A) and the dry substance (DS) concentration in the reactor (FIG. 2B).

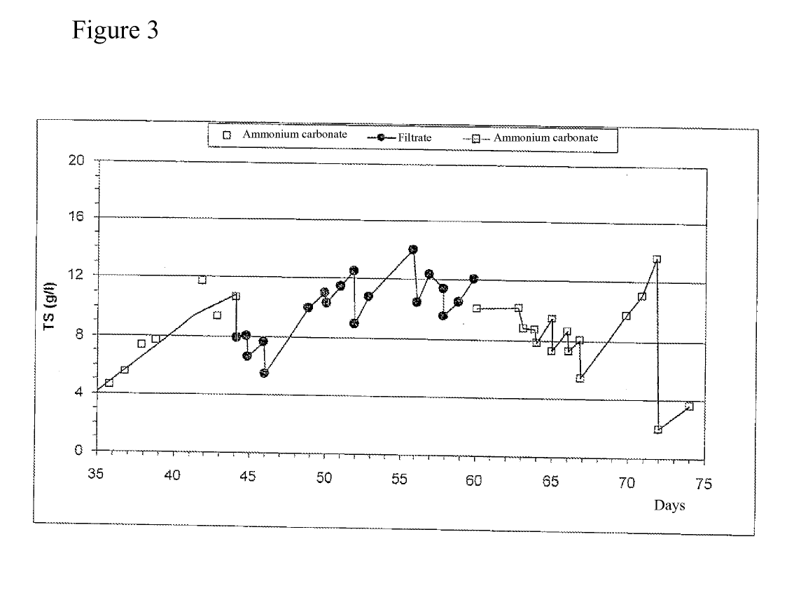

FIG. 3 illustrates the growth of Phaeodactylum tricornutum in a flatplate airlift photobioreactor (FPA) reactor at a light intensity of 400 μE m⁻²s⁻¹.

### EXAMPLE

### Device for the Conduct of the Method According to the Invention

An exemplary embodiment of the device (100) according to the invention is illustrated in FIG. 1. The device comprises a bioreactor (1), a filtration device (2) and a photobioreactor (3). The bioreactor (1) and the filtration device (2) are connected with each other by way of a line (12). Likewise, the filtration device (2) and the photobioreactor (3) are connected with each other by way of a line (23).

The bioreactor (1) can be designed for example as a fermentation tower and comprise devices for mixing the reactor content, for example a mixing device (11). Organic suspensions, for example wastewater, are anaerobic biologically treated in the bioreactor (1). The biogas generated in the process can be removed and used to generate energy. CO₂ produced from the biogas during the generation of energy can be supplied to the photobioreactor (3), whereby the CO₂ is bound through photosynthesis of the algae contained in the photobioreactor (3). The anaerobic biologically treated organic suspension, for example wastewater, is transported to the filtration device (2), for example a rotating disk filter, a polymer filter or a ceramic depth filter, via the fluid supply line (12). There, it is subject to further treatment that includes the removal of solid matter. The solid matter, also referred to as concentrate or fermentation residue, can either be resupplied to the bioreactor (1) via the supply line (24) or it is removed from the system via the discharge line (25). The filtrate is supplied to the photobioreactor (3) via fluid supply line (23). Algae, for example microalgae, are cultivated in the photobioreactor (3). The photobioreactor (3) can for example be a flat-plate airlift (FPA) reactor, featuring a special design with static mixers (31) that allow greater productivity at high cell concentrations.

The supplied filtrate is used as medium by the algae cultivated in the photobioreactor (3). This results in particularly good algal growth and at the same time to the further purification of the filtrate, because phosphate and nitrogen are removed from it.

The algae can be used to generate biomass or specific raw materials.

Analysis of a Filtrate Sample from an Anaerobic Process

A filtrate sample from a two-stage high-load fermentation for sewage sludge (TBB 6.12.08/29.4.08) was analyzed for its phosphate, ammonium nitrogen and COD content. The analysis yielded the following values:
PO4-P: 26.25 mg/L
Ammonium nitrate: 855 mg/L
COD: 200 mg/L
pH: 7.8
Water hardness 14.2 ° dH
Ca: 71.8 mg/L
Mg: 17.8 mg/L

With an average NH₄ demand of 30 mg/OD (optical density) for the growth of the microalga species Phaeodactylum tricornutum, a rise in the OD by 7.8 units per day can be achieved in the algal culture with the use of wastewater filtrate as media component and with a 20% medium exchange.

Cultivation of Microalgae with Different Known Nitrogen Sources Compared to a Filtrate Sample from an Anaerobic Process

Microalgae of the species Phaeodactylum tricornutum were cultured in an FPA photobioreactor. The nitrogen source available to the microalgae was changed at different points in time. During the first nine days, ammonium chloride was used as nitrogen source. From day 10 to 43, ammonium carbonate was used as nitrogen source. From day 44 to 60, the filtrate sample from an anaerobic process mentioned above was used as nitrogen source. From day 61 to 74, ammonium carbonate was used as nitrogen source. Urea was also used as nitrogen source.

The production rates of the algal culture were measured across different time periods.

The test results were summarized in FIGS. 2 and 3. The polynomial trend line of the measuring points for ammonium carbonate is drawn in FIGS. 2A and 2B. The measuring points for the wastewater are above said trend line.

As demonstrated, the best results were achieved with the use of the filtrate sample of an anaerobic process as nitrogen source. With the use of the filtrate sample, it was possible to achieve a high biomass productivity and at the same time a high dry substance concentration, even though the relative light availability was fairly low.

1. Bioprocess engineering method comprising the step: supply of at least one aqueous phase of at least one anaerobic biologically treated organic suspension as media component to an algal culture.

2. Method according to claim 1, wherein the aqueous phase is separated from the anaerobic biologically treated organic suspension in an additional step before being supplied to an algal culture.

3. Method according to claim 1 or 2, wherein the aqueous phase is a filtrate.

4. Method according to any one of the preceding claims, wherein the aqueous phase is derived from a methane fermentation process.

5. Method according to any one of the preceding claims, wherein the anaerobic biologically treated organic suspension and/or the aqueous phase are filtered at least once by means of a rotating disk filter, a polymer filter or a ceramic depth filter in the additional step before being supplied to the algal culture.

6. Method according to any one of the preceding claims, wherein the anaerobic biologically treated organic suspension and/or the aqueous phase are filtered at least once by means of a dynamic rotating disk filter, a dynamic polymer filter or a ceramic depth filter in the additional step before being supplied to the algal culture.

7. Method according to any one of the preceding claims, wherein the anaerobic biologically treated organic suspension and/or the aqueous phase are microfiltered or ultrafiltered in an additional step before being supplied to the algal culture.

8. Method according to any one of the preceding claims, wherein the anaerobic biological treatment is performed in an additional step as single-stage or two-stage methanization before the aqueous phase is supplied to the algal culture.

9. Method according to any one of the preceding claims, wherein the anaerobic biological treatment is performed in an additional step as methanization cascade before the aqueous phase is supplied to the algal culture.

10. Method according to any one of the preceding claims, wherein the untreated organic suspension is sewage sludge, organic waste, wastewater from the food industry, effluent from municipal wastewater and/or wastewater from the fermentation of renewable raw materials.

11. Method according to any one of the preceding claims, wherein the total medium used for the algal culture contains at least 50% by weight of the aqueous phase as media component.

12. Method according to any one of the preceding claims, wherein trace elements are added to the aqueous phase.

13. Method according to any one of the preceding claims, wherein the algal culture is a microalgal culture.

14. Method according to any one of the preceding claims, wherein the algal culture is a Phaeodactylum tricornutum culture, or a Haematococcus pluvialis culture, or a Chlorella sorokiniana culture, or a Chlorella vulgaris culture, or a Platymonas subcordiformis culture, or a Tetraselmis suecica culture, or a Nannochloropsis oculata culture, or a Nannochloropsis limnetica culture, or a Phormidium sp. culture, or a Pseudoanabaena sp. culture, or a Dunaliella sp. culture, or a Monodus subterraneus culture, or an lsochrysis sp. culture.

15. Method according to any one of the preceding claims, wherein the algal culture is cultivated in a photobioreactor.

16. Method according to any one of the preceding claims for the manufacture of biomass derived from an algal culture and/or for the manufacture of valuable substances.

17. Method according to any one of the preceding claims for the treatment of aqueous phases of anaerobic biologically treated organic suspensions.

18. Use of an aqueous phase of an anaerobic biologically treated organic suspension as media component of an algal culture.

19. Use of an aqueous phase of an anaerobic biologically treated organic suspension to improve the growth conditions of algae in photobioreactors.

20. Use of algae for the treatment of an aqueous phase of an anaerobic biologically treated organic suspension, in particular of anaerobic biologically treated sewage filtrate.

21. Bioprocess engineering device (100), comprising a bioreactor (1) and a photobioreactor (3).

22. Device (100) according to claim 21, wherein the device comprises a filtration device (2).

23. Device (100) according to claim 22, wherein the filtration device (2) is a microfiltration device (2).

24. Device (100) according to claim 21 or claim 22, wherein the bioreactor (1) has a fluid supply line (12) to the filtration device (2) and the filtration device (2) has a fluid supply line (23) to the photobioreactor (3).

25. Use of a device (100) according to any one of claims 21 to 24 for the conduct of a method according to any one of claims 1 to 17.
